(19) United States
(12) Patent Application Publication
(10) Pub. No.: US 2012/0029455 A1
(43) Pub. Date: Feb. 2, 2012

(54) WOUND DRESSING, METHOD FOR THE PRODUCTION THEREOF, AND USE THEREOF FOR DRESSING WOUNDS

(75) Inventors: David Pérez-Foullerat, Madrid (ES); Sascha Casu, Hamburg (DE); Axel von Wolff, Hamburg (DE); Angela Sauerwald, Hamburg (DE)

(73) Assignee: BSN MEDICAL GMBH

(21) Appl. No.: 13/147,584

(22) PCT Filed: Feb. 2, 2010
(86) PCT No.: PCT/EP10/51253

§ 371 Date: Sep. 2, 2011

(30) Foreign Application Priority Data
Feb. 2, 2009 (DE) 10 2009 007 101.6

(51) Int. Cl. A61L 15/22; B32B 37/14

(52) U.S. Cl. 604/368; 156/60; 156/307.1

(57) ABSTRACT

The present application relates to a wound dressing, method of producing a wound dressing of this type and its use.

The present invention relates to a wound dressing, method of producing such wound dressings and their use.

For wound dressings and/or compresses for medical purposes numerous materials based on film, woven, knitted, non-woven, gel or foam materials are known and are also used in practice.

The following requirements are set and have to be met by a wound dressing for treating wounds that tend to a certain amount of fluid secretion: the wound dressing must exhibit sufficient absorbency for wound fluid, but at the same time it must exhibit sufficient wet strength. The wound dressing must prevent the penetration of foreign bodies (such as bacteria and dirt) into the wound and stop the wound fluids leaking into the area outside the wound dressing. The wound dressing should only adhere slightly to the skin surrounding the wound, i.e. it should not stick to the wound. The wound dressing not cause irritation in the covered tissue. The wound dressing should mould easily to contoured parts of the body. The wound dressing should be permeable to gases and water vapour. In addition the wound dressing should be able to be combined with medicinal products and/or wound healing active substances (such as bactericides or growth factors).

Such systems are described, for example, in U.S. Pat. No. 4,373,519 and U.S. Pat. No. 6,566,576 as well as in U.S. Pat. Nos. 5,409,472, 5,782,787, 3,972,328 and 5,395,305.

The wound dressing forming the basis of this application promotes wound healing through the formation of an advantageous moist wound environment.

For promoting the healing process the presence of at least part of the exudate is of primary importance so that a moist micro-environment in the direct vicinity of the wound can be maintained. Through the wound dressing only part of the exudate is removed from the wound environment. This means that in the immediate vicinity of the wound acertain moisture content is maintained which is beneficial to successful wound healing, through rapid granulation of the wound for example.

Through the removal of the excess exudate and the toxic components, the wound dressing prevents the exudate collecting at certain points and as a result of this the likelihood of bacterial accumulation and skin maceration increasing at these points. In this way the wound dressing reduces the risk of a bacterial secondary infection.

In the past, in order to promote the above-described formation of a moist wound environment, numerous wound coverings were developed which have an absorbent layer with a certain absorption capacity. Usually hydrophilic materials are present in the absorbent layer which can absorb the exudate. This means that the wound dressing can in certain circumstances remain on the wound for several days, whereby within this period optimum conditions for wound healing are ensured.

Wound dressings which have an adhesive layer and which are arranged in such a way that they are in contact with the surface of the wound are known from the prior art. However the use of adhesives with a high affinity to skin, such as acrylates for example, results in the wound dressing sticking to the wound, which means that tissue formed during healing is damaged by removing the dressing. Soft gels and elastomers—particularly those based on silicone or polyurethane—which are characterised by a preset, gentle adhesive behaviour, are known as being advantageous dressing materials in wound care. These materials can be hydrophilic or hydrophobic, do not stick to the wound or the wound bed and can be designed so that they adhere to the wound in a mild manner. This ensures painless removal of the wound dressing from the sensitive wound area. Such gels and/or elastomers are disclosed in the following patent documents, for example: EP-A-0 251 810, U.S. Pat. No. 4,921,704, U.S. Pat. No. 6,051,747, WO-A-2007/113597, WO-A-2007/113453, WO-A-2004/060412 as well as U.S. Pat. No. 5,336,695. Nevertheless, the use of these wound-tolerable and skin-friendly materials when laminating them with the required substrates causes difficulties which mainly consist in the fact that due to their not very pronounced cohesion behaviour, there may be technical problems in anchoring these materials to the relevant substrate. Further disadvantages lie in the limited selection of suitable dressing components and in the marked flow behaviour of the raw materials required for production as long as these are not yet in a crosslinked state.

The wound dressing disclosed here has structure with optimised skin adhesion, residue-free and painless removal (from the wound area) and form stability and has an excellent fluid-absorbing ability. The swelling and deformation of the absorption layer or absorption body caused by absorption of the exudate can be reduced, which minimises the loosening or separation of the layer in contact with the body from the skin through curling, particularly at the edge of the wound dressing. This loosening can lead to leakage of the wound dressing which allows the penetration of bacteria, or in the case of laminated wound dressings can lead to delamination of the components. By contrast, the wound dressing described here cannot tend to said curling or delamination even after considerable absorption of exudate.

The wound dressing also allows the wound exduate to be transferred unhindered into the interior of the absorption body where it is collected. However, undesirable contact between the absorption body and the wound is prevented.

Furthermore, the wound dressing does adhere to the moist areas of the wound, but only to intact skin. This makes it possible for the wound dressing to be removed from the wound without trauma. The adhesion with regard to the skin can be set in such a way that it meets the special requirements of the application in question.

The wound dressing has the advantage that is can be easily and cost-effectively produced and the wound dressings produced with the manufacturing process open up further scope with regard to the characterising parameters and choice of materials.

The wound dressing is therefore eminently suitable for wound care.

In accordance with one form of embodiment a wound dressing is provided which is essentially formed of a three-layer laminated wound care system. Accordingly the wound dressing comprises a wound contact layer which has openings and which is attached to an air and water-permeable carrier, and a hydrophilic absorption layer which adjoins the carrier.

In a further form of embodiment the present application relates to a wound dressing 10 comprising a gel or elastomer layer 12, which has openings 34 and is connected to a fluid and air-permeable carrier 19, and an absorption layer 14 which adjoins the carrier 19.

In one form of embodiment the present application relates to a wound dressing 10 which has a fluid-impermeable, gas and water vapour-permeable backing layer (i.e. the layer facing away from the body).

In a further form of embodiment the present application relates, among other things, to a wound dressing 10, which has a carrier layer 19 for the gel or elastomer layer 12, whereby the carrier layer is made of a fibre-like material.

In another form of embodiment the present application relates to a wound dressing 10, in which elements, such as, for example, fibres, of the fibre-like carrier layer 19 penetrate into the gel or elastomer layer 12 up to a depth of 5 to 100 μm.

In a further form of embodiment the present application relates to a wound dressing 10, in which the wound contact layer 12 embodies a hydrophobic, crosslinked silicone gel or silicone elastomer.

In a further form of embodiment the present application relates to a gel or elastomer 12 which adheres to the skin.

In another form of embodiment the present application relates to a wound dressing 10, in which the fibre-like material of the carrier 19 embodies an elastic melt-blown web.

In a further form of embodiment the present application relates among other things to a wound dressing, which on the side facing the skin has a separable protective film, e.g. in the form of a pull-off film, whereby the protective layer is removed before using the dressing.

In a further form of embodiment the present application relates among other things to a wound dressing which has an absorption layer with strongly absorbent hydrophilic materials.

In a further form of embodiment the present application relates among other things to a wound dressing which has devices for fastening the wound dressing on the side facing the skin.

In a further form of embodiment the present application relates among other things to a wound dressing in which the thickness at the edge of absorption layer is less than the thickness of the absorption layer between the edges.

The present application also relates to a method of producing a wound dressing comprising the steps
a) Laminating an absorption layer with a carrier.
b) Laminating the partially crosslinked gel or elastomer layer, which has openings, with the laminated carrier

In addition, the present application relates to a wound dressing for use in a procedure for treating a wound.

Further forms of embodiment are evident from the invention and the patent claims.

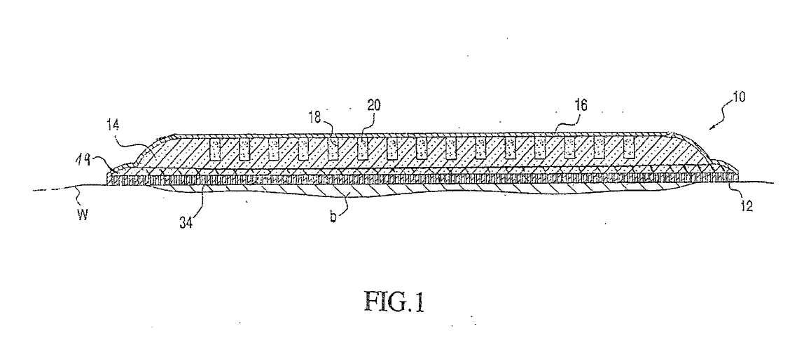

FIG. 1 shows a cross-section of a form of embodiment of the wound dressing over a wound.

The at least three planar components 12, 14, 19 forming the basis of the wound care system 10 of the present application consist of an absorption layer 14, a fibre-like carrier material 19 and wound contact layer comprising silicone-based gel or elastomer 12. The individual layers can be made up of mixtures of various materials and they do not necessarily have to be homogeneous. Thus, absorption layer 14 can, for example, be a foamed polymer material in which particles 20 can be embedded which absorb the exudate particularly well and even under the application of pressure essentially do not release the taken-up exudate. Such particles 20 can, for example, be built up of so-called superabsorbers. In addition, particles can also be embedded (not shown in FIG. 1) in the gel or elastomer layer located on the side of the wound care system facing the skin, said particles being required for controlling the mechanical properties, the hydrophilia or the adhesion. These particles can, for example, consist of organic or inorganic compounds which can be of a polymer nature.

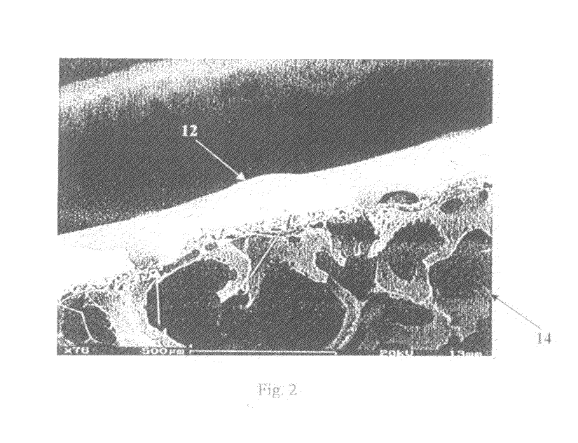

FIG. 2 shows an REM image in backscattered electron mode of a cross-section of a form of embodiment of the wound dressing enlarged multiple times. FIG. 2 shows an REM image of a wound dressing in accordance with the invention which has an elastomer layer 12 which is provided with openings 34, whereby said layer is connected to a fluid and an air-permeable carrier 19 adjoining which is an absorption layer 14.

In addition the wound care system and/or the wound dressing can comprise additional components or layers/coatings. Thus, in the overwhelming majority of cases it is considered desirable for a wound dressing to be available which always has a dry outer side (i.e. side facing away from the wound W or the wound contact layers 12). The penetration of dirt and bacteria into the wound dressing from outside and in the worst case reaching the wound should also be prevented. This aim can be achieved by, for example, applying a fluid-impermeable continuous protective film 16 (hereinafter also referred to as backing film or film), whereby in a practical manner the backing film or film 16 is water vapour-permeable. This layer which, as has been mentioned, should typically be impermeable to bacteria, adjoins the distal surface of absorption layer. In an advantageous form of embodiment the film is only bound to the distal surface of the absorption layer in a manner so that the film 16 cannot penetrate into the pores, cells or other intermediate spaces. The backing layer can be transparent to allow the level of filling or moisture in the wound dressing or the status of the wound to be assessed without having to remove the dressing. The backing layer can be filled with colouring agents. In general the film has a thickness of 10-500 μm and typically 15 to 45 μm, whereby film thicknesses of 30 +/−5 micrometres are used in particular.

Films of this type are known from the prior art and comprise, for example, polyurethane-based films, such as a polyurethane film supplied by Exopack Advanced Coatings (Wrexham, UK) under the product name INSPIRE®, or elastomer polyesters or mixtures of polyurethane with polyesters and/polyvinyl chloride and polyetheramide block copolymers. Alternatively the backing layer can be a water-repellent and water vapour-permeable polyurethane foam with essentially closed cells, such supplied, for example, by Scapa (Greater Manchester, UK) under the product name Medifix. For the purposes of the present application a polyurethane film is used as these films have good elastic properties and, in particular, exhibit form fitting properties as well as a high level of stretchability

In themselves suitable films have a moisture-vapour transmission rate (MVTR) of 500 to 14600 gm⁻²/24 hours, typically 1000 to 2700 gm⁻²/24 hours at 38° C. Higher MVTR values can be advantageous in order to delay the saturation point of the wound dressing in strongly secreting wounds. Low MVTR values can be beneficial in assuring a moist micro-environment around the wound in the case of low-secretion wounds.

On the distal surface of the absorption layer the backing layer can be laminated in any known way. For example lamination can take place by means of heat or ultrasound or by means of an additional continuous and discontinuous adhesive layer arranged between the backing layer and the absorption layer.

Depending on the intended purpose of use it may be necessary to use film of a different thickness or to combine several layers/film. Thus, it may be advantageous to provide the above-described layers 16 with a carrier layer (not shown in FIG. 1) in order to guarantee a particular mechanical strength and thus prevent wrinkling of the backing layer. In general the thickness of the entire layer—i.e. the film and, if applicable, the carrier and additional layer(s)—should be in a range of 5 to 2000 micrometres and typically in a range of 5 to 1000 micrometres. The layer and/or the outermost film should, for practical purposes, exhibit a low coefficient of friction and, for example, not catch on textiles or clothing, rub on them or negatively interact with textiles in general.

The layer 12 next to the skin is, for example, formed of a hydrophobic layer based on a silicone gel or silicone elastomer (therefore hereinafter referred to as the “silicone layer”), which has openings. This perforated layer should initially separate the wound W from the absorption layer 14. This layer must remain mechanically stable and be able to be removed from the wound area with as little residue as possible—even after longer contact with the exudate.

Layers in contact with the wound can be made of silicone gels or silicone elastomers. These do not adhere to the wound, but can be designed so that they exhibit variable adhesion to the dry skin surrounding the wound. In cases where the wound dressing has a silicone layer which significantly adheres to the dry skin as the adhesive force is evenly distributed through the silicone layer, no separation of epidermal cells or damage to the wound is observed (this is the main drawback of hard adhesives, such as adhesives based on acryl derivatives, which exhibit high specific adhesion). Silicones of this type are completely immobile and are not affected by heat or bodily exudates. These properties make it possible to remove the wound dressing from the wound and from the wound surroundings without causing pain to the wearer of the dressing or causing trauma.

The silicone gels or silicone elastomer forming the layer in contact with the wound can be made of two-component initial mixtures of silicones which after being brought into contact harden to the required extent. Such systems are known from the prior art, for example from EP-A-0 251 810, EP-A-0 300 620 or U.S. Pat. No. 4,921,704. The systems described therein essentially comprise a component A, consisting of at least one vinyl-substituted polydimethyl siloxane as well as a platinum catalyst. Component B contains polydimethyl siloxanes with hydrogen atoms bonded directly to the silicon atom. In accordance with the prior art additives such as pigments, inhibitors or filling agents, such as silicon dioxide, can be included in both components if desired.

The combining of the two components results in activation of the crosslinking reaction of other functionalised polymethyl siloxanes which eventually leads to hardening.

The time required for the desired hardening depends on various factors, such as, for example, the reaction temperature or the catalyst concentration or, as the case may be, the presence of inhibitors.

Even through in the above-described method essentially very similar initial components (educts) can be used, the properties of the fully hardened silicone layer can be influenced in various different ways—e.g. through varying the ratio of the components A and B, by modifying the stoichiometric ratios of the groups responsible for the crosslinking—such as the vinyl groups and silicon-hydrogen groups, through the molecular weights of the polysiloxanes used or through the concentration of the filling agent(s) used. In this way silicon gels can be made available that are soft, very adhesive and not friable and exhibit significant adhesion to the skin.

On the other hand silicone elastomers can be reinforced with filling agents which gives them a higher consistency. In addition, they are more robust, harder with no/little stickiness and do not adhere to the skin—at least not to an extent sufficient to ensure permanent attachment to the skin.

The cited silicone gels are commercially available from the company NuSil Technologies (Carpinteria, US) under the product name MED-6345 or MED-6340 or from the company Dow Corning GmbH (Wiesbaden, DE) under the product name Dow Corning® 7-9800 or from the company Wacker Chemie GmbH (Munich, DE) under the product name SilGel.

Silicone elastomers are also commercially available—e.g. under the product name MED-6305 from the company NuSil Technologies (Carpinteria, US), under the product name Silbione RTV-4511 from the company Bluestar Silicones, or under the product name Silastic MDX4-4210 from the company Dow Corning GmbH (Wiesbaden, DE).

In addition, numerous other crosslinked polymers and pressure-sensitive adhesive masses can be used as a wound contact layer—such as polyorganosiloxanes containing silanols which are crosslinked in the presence of tin octoate and/or produced by heating. The silicon polymer can desirably have substituents, where polyethylene glylcol or polyurethane count as possible substituents.

The properties of the silicone elastomer or gel can also be controlled by mixing several silicon elastomers or silicone gels, such as the silicine elastomer MED 4905 and silicone gel MED 6340.

The silicone layer is typically perforated in order to allow adequate transfer of moisture (exudate) and air—air/oxygen away from the wound and to the wound. The number and geometrical arrangement of the perforations 34 is generally determined by the intended purpose of use of the wound dressing and can be adjusted irrespective of on the structure of the other components of the wound dressing.

Although silicone-based systems are preferred, there are other alternatives available with regard to the use of hydrophobic or hydrophilic polyurethane derivatives of a gel or elastomer nature, such as LEVAGEL, obtainable from Bayer AG (Leverkusen, DE). In addition hydrophilic polyurethane gels, such as described in U.S. Pat. No. 6,191,216, U.S. Pat. No. 6,566,575 and EP-A-0 271 292 can be used. Another alternative consists in the use of partially or fully hardened hydrophilic hydrogels—for example on a acrylate or monosaccharide basis—or in the use of skin-adhering elastomer hydrocolloid masses.

The perforated gel or elastomer 12 is applied to a carrier 19 in a subsequent step. The carrier 19 is air and fluid-permeable and itself has no fluid absorbing ability worthy of note. If necessary the carrier 19 can also be perforated, whereby the perforations on in the carrier layer and the gel or elastomer layer may or may not coincide. The perforations in the carrier layer can be produced in an additional processing stage. Alternatively the perforations in the carrier layer can be produced at the same time as the perforations in the gel or elastomer layer. In accordance with the present application the carrier and/or the carrier layer 19 consists of a non-woven or melt-blown material which has an irregular structure formed of small fibres. Particularly suitable for this are plastic or thermoplastic melt-blown webs or non-wovens which meet these requirements. The use of conventional fibre materials is generally more cost-effective than the production of woven or knitted materials. Non-wovens can be produced by many different methods, for example the dry method, the spunbonding method or wet method. If required a serious of refining stage can follow on from these methods. The reinforcement of non-wovens for medical applications in wound dressing and compresses is carried out, for example, thermally or mechanically so that the finished non-woven comes into no further contact with processing or auxiliary chemicals during production. As a result material produced using these methods are particularly suitable for use in medical products, such as, for example, use of the wound dressing 10.

The thickness of the fibre-like carrier layer lies in a range of 5 to 250 μm, typically in a range of 5 to 150 μm and particularly typically in a range from 50 to 150 μm. However, depending on the purpose of use, narrower ranges from 10 to 30 μm can be considered. A suitable carrier has an elastomer nature and is deformable in order to be able to mould to both the physical contours of the wearer as well as to the swelling body of the absorption layer. This can prevent curling of the wound dressing and thus reduce the risk of separation from the wound.

Useable carriers are disclosed in U.S. Pat. No. 5,230,701 for example. Typically non-woven thermoplastic elastomer webs made of polyurethane are used, which are for example available commercially from the company Freudenberg Vliesstoffe KG (Weinheim, DE) under series name XO of the brand name Vilene®. In addition, other non-wovens, made of cellulose, polyolefins, polyesters or polyamides, can be used.

Depending on the materials used the lamination of the elastomer or gel layer and the carrier as well as the lamination of the carrier and the absorption layer can take place in various ways, whereby the carrier layer and elastomer or gel layer should also exhibit sufficient permeability for the exudate and air after lamination. Advantageously, during lamination the fibres of the carrier 19 partially penetrate into the elastomer or gel (from 5 to 100 μm), which may also have a positive effect on the strength of the bond. The degree of hardening of the gel or the elastomer during lamination with the carrier layer can be set so that the partially crosslinked gel or elastomer layer can flow into the microscopic empty spaces between the fibres. In this way laminates can be produced which exhibit very good anchoring of the elastomer or gel in the carrier without the need to use additional adhesives or primers.

The wound dressing 10 in accordance with the invention has an absorption layer 14, comprising at least one hydrophilic material.

The absorption layer 14 can among other things consist of a foamed material (foam) which has open pores and cells. The pore size is uncritical with respect to the other layers; suitable pore sizes are in a range of 30 to 700 μm. The foamed material of the absorption layer can also exhibit a gradient with regard to the cell sizes along the thickness of the absorption layer. Such absorbent foamed materials can be produced from various foamable materials, such as, for example: polyurethane, carboxylated celluloses, butadiene-styrene copolymers, carboxylated butadiene-styrene rubbers, foamed polyesters, foamed hydrophilic epoxides or polyacrylates, hydrophilised silicones or foams based on ethylene vinyl acetate (EVA).

Also suitable are woven or non-woven materials capable of absorption, such as woven materials of cellulose fibres, cellulose flakes or matrices based, for example, on polymer fibrils such as alginates or chitosans.

In a typical form of embodiment the absorption layer 14 is embodied by a hydrophilic polyurethane foam such as, for example, a polyurethane foam commercially available under product name L00562-B from the company Rynel Inc. (Wiscasset, US) or under the product name VIVO MCF.05 from the company Corpura (Etten-Leur, Netherlands).

Through further processes it is possible to subsequently increase the hydrophilia of the foamed materials to be used if desired or necessary. With this method the tendency of the exudate to penetrate into the foamed material can be increased. However it should be ensured that the hydrophilia of the foamed material in question does not become so great that the exudate remains in the foamed materials and is no longer transported to the absorbent particles which may be arranged or present within the foamed material. In fact the hydrophilia of the foamed material can be adjusted with additives in such a way that the surface tension is minimised to allow simple passage of the fluid into all foam cells whereby a sufficient moisture content on the wound can be maintained.

However the absorption layer 14 does not necessarily have to be based on a compact piece of foamed material. Thus, in a further form of embodiment the absorption layer 14 can comprise a porous woven or non-woven material. For example, the absorption layer 14 can be a voluminous, loosely formed web consisting of very short cellulose fibres arranged in random or non-random sequence with a cushion of cellulose flakes, chitosan flakes or polymer fibre matrices.

The thickness of the absorption layer is generally within a range from 0.5 millimetres to 20 millimetres and typically between 3 millimetres and 5 millimetres. Depending on the type of use and the absorption capacity requirement, other thickness ranges may be advantageous.

The absorption layer 14 can typically contain one or more so-called superabsorber(s) in the form of granulate, flakes or powder. Designated by the term superabsorber (occasioanally also called “super slurper”) are polymers characterised by their extreme absorbency, i.e. ablity to take up many times their mass (e.g. up to 30-800 times) of water. Even under moderate pressure on the superabsorber this water is not released. This ability to take up water is based on the powerful interaction of the water molecules with hydrophilic groups of the superabsorber, more particularly with ionic groups or groups capable of hydrogen bridge binding. Large numbers of these superabsorbers are known from the prior art and in terms of their structure can generally be divided into three categories: starch graft copolymers, crosslinked carboxymethyl cellulose derivatives and modified hydrophilic polyacrylates. Examples of such absorbent polymers are hydrolysed starch acrylonitrile graft copolymers, neutralised starch acrylic acid graft copolymers, saponified acidic vinylacetate-acyrlic acid ester copolymers, hydrolysed acrylonitrile copolymers or acrylamide copolymers, modified crosslinked polyvinyl alcohols, neutralised self-crosslinking polyacrylic acid, crosslinked polyacrylate salts, carboxylated cellulose as well as crosslinked isobutylene maleic acid anhydride copolymers.

Hydrophilic polymers in the form of particles with superabsorber properties are described, for example, in U.S. Pat. No. 4,102,340. In particular absorbent materials such as crosslinked polyacrylamides are used for this. Suitable absorbent superabsorbing particles consist of crosslinked partially neutralised polyacrylic acid and are used in some of the forms of embodiment described here.

The superabsorbing particles 20 are produced, for example, from a starch polyacrylate graft copolymer hydrogel, and are available in powder form from Hoechst-Celanese (Portsmouth, US). Other particles with superabsorbing properties are commercially available under the brand name SANWET (available from Sanyo Kasei Kogyo Kabushiki Kaisha, JP) as well as DEM SUMIKA GEL (available from Sumitomo Kagaku Kabushiki Kaisha, JP) which comes in the form of an emulsion and after polymerisation is present in the form of spherical particles, as well as superabsorbers commercially available under the name FAVOR (e.g. from Evonik Industries AG, Essen, DE).

The superabsorber particles are for example used in the form of grains or flakes in order to be able to provide a large hydrocolloidal surface area. In the dry state the size of the superabsorber particles 20 is usually within a range of 1 to 1000 micrometres and typically within a range of 100 to 900 micrometres. The particles which are not soluble in the conditions prevailing under the wound dressing typically exhibit a water absorption capacity of more than 0.5 per gram of dry particles.

In accordance with a further form embodiment the absorbent material can be a hydrophilic gel which swells up after coming into contact with water. Hydrophilic gels generally lack a cellular or empty (hollow) internal structure. Such gels are usually present in a solid or semi-solid state. Hydrophilic gels in the sense of this application are understood as hydrocolloids, hydrogels and combinations thereof—insofar as they are physiologically tolerable. Suitable hydrophilic gels are disclosed in US patent specification U.S. Pat. No. 6,566,575 and are also commercially available.

The superabsorber particles can be homogenously distributed in the absorption layer or they can be arranged within the cells of the polymer form. To produce these structures the particles can be mixed with the initial materials for producing the foam before foaming, or they are placed in cells provided to hold them in a production stage following foaming.

In accordance with a further form of embodiment of the wound dressing the absorption layer—for example the foam material—can have holding containers 18 in which superabsorber particles, for example, are to be found. The holding containers 18, formed for example by recesses in the absorption matereial, can be of any geometrical form, for example cubes, cones or cylinders.

Such holding containers 18 can be of a uniform, predetermined shape and size and typically extent over the distal area of the 14, whereby the holding containers 18 can be arranged in any form, e.g. is grid-like structure. The geometrical dimensions and, in particular, the volume of each holding container are determined by the relevant requirements, e.g. in accordance with the quantity and size of the superabsorber or gel to be held. An example of such a system is disclosed in WO-A-2004/060412.

In accordance with one possible form of embodiment the holding containers for the superabsorber particles are distributed over the entire thickness of the absorption layer and form channels which are filled with superabsorber particles. The carrier layer forms a barrier which holds back the superabsorber particles in the absorption layer 14 thus preventing them from reaching the wound.

In a further form of embodiment of the wound dressing, the absorption layer 14 can comprise several separate sections of the absorbent material. The absorbent material can be arranged in compartments. Such compartment can contain a superabsorbing polymer present in the form of a granulate or corresponding polymer which is present in the form of flakes or powder. The individual particle can however also be freely moveable within the absorption layer whereby they preferably migrate towards the distal surface.

The superabsorber or the absorbing gel can also be applied as a further layer on the distal surface of the absorption layer.

The advantage of the described forms of embodiment in general lies in the fact that the amount of fluid that can come into contact with the wound can itself be minimised and increased through absorption in the part of the absorbing layer facing away from the wound. In this way the time the wound dressing remains on the patient's body is increased and/or the maceration risk for the skin surround the wound is reduced.

The wound dressings in accordance with the invention have the further advantage that they can be easily produced with known standard methods.

A wound dressing in accordance with the present invention can, for example, be produced as follows:

In a first step the carrier layer is laminated with the hydrophilic and/or open cell or semi-open cell absorption layer. Depending on the material used the lamination can be carried out different ways, e.g. through the use of heat of radiation, e.g. UV radiation or ultrasound. However, when selecting the lamination method it must be ensured that neither of the two layers lose their desired properties in terms of absorption, exudate permeability and air permeability. When using polyurethanes thermal lamination is more particularly used. It is also possible to directly coat the carried layer with the initial materials required for producing the absorption layer and have the foaming process carried out afterwards, whereby firm anchoring of the two layers to each other is achieved.

In a second step, to produce the silicone layer a hardenable silicone mixture is applied to a carrier band which has devices for producing perforations such as blunt needles which extend through the silicone layer. Through suitable measures, e.g. heating, the silicone mixture is partially hardened resulting in a silicone layer which has the specified openings. Expediently the silicone layer has a thickness of 10 to 250 micrometres, preferably between 60 and 150 μm.

Thereafter, in a third step the proximal surface of the carrier of the laminate resulting from the first step is brought into contact under pressure with the partially hardened silicone layer, preferably still on the transport band on which the partially hardened silicone layer is to be found, and then a further hardening step is carried out with which the silicone layer is attached to the laminate produced in the first step, resulting in very good anchoring of both layers. The open structure of the fibre-like carrier layer is associated with a very large contact surface area which is available for forming a contact between the carrier layer and absorption layer. The conditions for producing the contact and/or anchoring can be selected so that fibres of the carrier material penetrate into the silicone layer, which leads to efficient connection of both layers without having to use an additional adhesive or primer.

Production of the complete wound dressing can be followed by further processing steps, such as, for example, sterilisation or packing in a sterile storage container.

If desired, the wound dressing 10 can also contain pharmaceutically active substances, which, for example sterilise the wound or promote healing such, for example, antibacterial and/or antimycotic active substances and/or growth factors. In addition, haemostatic as well as anti-inflammatory active substances can be included in the wound dressing. In addition, soaps and possibly deodorising agents such as active charcoal, for example, can be used. All active substances that can be considered are sufficiently well known from the prior art and can incorporated into the elastomer or gel layer 12 and/or into the absorption layer in a practical manner. If desired or if it appears expedient for other reasons, these can also be contained in an additional, separate layer.

The wound dressing 10 can have devices for fastening the wound dressing to the wound area or on the body (not shown in FIG. 1). This device can be formed by one or more area(s)—incorporating the elastomer or gel layer at the peripheral edge section—or the peripheral edge, which have an adhesive for attaching to skin.

Examples of typical forms of embodiment can be gleaned from WO-A-2005/034797 and WO-A-2006/127292. The adhesive in the area adjoining the skin can be embodied by any medically tolerated adhesive, such those based on acrylates, rubbers, polyurethanes or silicones.

Preferably this adhesive is a pressure-sensitive silicone, such as, for example, an adhesive silicone commercial available from NuSil Technology (Carpenteria, US) under product name MED-1356, or a sticky silicone gel which is also commercially available from NuSil Technology (Carpenteria, US) under the product name MED-6345.

The method described above as an example thus results in a laminated wound dressing which exhibits the advantages set out in the introductory section.

### EXAMPLE

A laminated wound dressing is produced as follows:

Laminated onto one side of a hydrophobic foam layer with thickness of 5 mm, an absorption capacity allowing free swelling of 16 g/g foam (DIN 13726-1:2002) and density of 96 kg/m³ is a fibrous polyurethane non-woven with a thickness of 0.15 mm, with an air permeability of 520 l/m² s at 100 Pa and 40 g/m² weight. Lamination takes place by means of a commercially available industrial band laminator by the company Herbert Meyer GmbH in Rotz, Germany, at a temperature setting of 175° C. The exposure time is approximately 6 seconds with a gap setting of 3 millimetres. The laminate is thus firmly bonded whereby the fibre structure and air and water permeability of the PU non-woven are preserved.

In a separate step a non-crosslinked mixture is prepared of a commercially available 2-component addition crosslinkable silicone gel, with a penetration value of 3 mm (after hardening for 30 minutes at 140° C. measured with a GCA Precision Penetrometer 19.5 g cone, 635 mm foot diameter, 15 seconds). The silicone mixture is applied evenly with trimmer beams to a metal band with needles, whereby the height of the needles is greater than the layer thickness of the silicone layer. The metal band carrying the silicone layer is then heated to a temperature of 120-180° C. in order to accelerate the crosslinking of the silicone. During this, by means of a pressure roller the foam-non-woven laminate is pressed with its polyurethane-non-woven side onto the partially crosslinked silicone mixture. The silicone layer of the thus resulting 3-layer laminate continues to be heated on the metal band in order to complete the crosslinking.

On completion of the crosslinking the 3-layer laminate is removed from the metal band. Through the pattern of the needles on the metal band corresponding gaps/openings have been produced in the silicone layer.

The three-layer laminate is pliable and easily adaptable. The silicone layer is securely anchored on the fibrous non-woven material. An aqueous solution can pass through the silicone and polyurethane non-woven layers without hindrance and is then absorbed in the foam if a beaker with an aqueous solution (solution A, DIN 13726-1:2002) is placed upside down on the silicone side of the laminate.

1. Wound dressing comprising:
a gel or elastomer layer which has openings and which is anchored to a fluid and air-permeable carrier laver, comprising a non-woven, through the penetration of the fibers of this non-woven into the gel or elastomer layer without the use of additional fixation adhesives, and
an absorption layer which adjoins the carrier.

2. Wound dressing in accordance with claim 1 characterised in that the gel or elastomer layer is a hydrophobic crosslinked silicone.

3. Wound dressing in accordance with claim 1 characterised in that the gel or elastomer layer adheres to the skin.

4. (canceled)

5. Wound dressing in accordance with claim 1 characterised in that the carrier layer is perforated.

6. Wound dressing in accordance with claim 1 characterised in that the fiber-like material is an elastic melt-blown web.

7. Wound dressing in accordance with claim 1, characterised in that fibers of the carrier layer penetrate from 5 to 100 μm into the gel or elastomer layer.

8. (canceled)

9. Wound dressing in accordance with claim 1, characterised in that the absorption layer comprises at least one hydrophilic material.

10. Wound dressing in accordance with claim 1, characterised in that that the absorption layer comprises a polyurethane foam.

11. Wound dressing in accordance with any one of claims 1 to 10, characterised in that the wound dressing comprises devices for fastening of the wound dressing on the side facing the skin.

12. Method producing a wound dressing in accordance with any one of claims 1 to 3, 5 to 7, and 9 to 10 comprising:
a. Lamination of the absorption layer with the fluid and air-permeable carrier layer; and
b. Lamination of the gel or elastomer layer which has openings with the laminated carrier layer.

13. Method producing a wound dressing in accordance with claim 12 characterised in that during the lamination the gel or elastomer layer is initially partially crosslinked to the carrier layer and the crosslinking is completed in an additional subsequent step.

14. Wound dressing in accordance with any one of claims 1 to 3, 5 to 7, and 9 to 10 for use in a method of treating a wound.

15. Wound dressing in accordance with any one of claims 1 to 3, 5 to 7, and 9 to 10, characterised in that the wound dressing comprises a moisture-impermeable, water vapor-permeable backing layer.
